(19) United States
(12) Patent Application Publication
(10) Pub. No.: US 2019/0201072 A1
(43) Pub. Date: Jul. 4, 2019

(54) BIOLOGICAL STIMULATION TOOL

(71) Applicant: Yoko SHIRAISHI, Toyonaka-shi, Osaka (JP)

(72) Inventor: Yoko SHIRAISHI, Toyonaka-shi, Osaka (JP)

(21) Appl. No.: 16/314,546

(22) PCT Filed: Jun. 29, 2017
(86) PCT No.: PCT/JP2017/023845

§ 371 Date: Dec. 31, 2018

(30) Foreign Application Priority Data
Jul. 4, 2016 (JP) 2016-132836
Aug. 3, 2016 (JP) 2016-152895

(51) Int. Cl. A61B 18/08; A61F 7/00

(52) U.S. Cl. CPC A61B 18/082; A61B 2017/00084; A61F 2007/0003; A61F 7/007

(57) ABSTRACT

The present invention provides a biological stimulation device that can stimulate muscles to recover tension with no adverse effects on living bodies. Since the biological stimulation device of the present invention includes a stimulation device main body, a heating part integrally provided at a distal end surface of the stimulation device main body, and an electrode part integrally provided at the distal end surface of the stimulation device main body, firing is effectively generated in activated nerve cells. Further, the muscles are stimulated to provide tension to the muscles, so that it is possible to effectively reduce or remove wrinkles and the like appearing on the skin.

### TECHNICAL FIELD

The present invention relates to a biological stimulation device.

### BACKGROUND ART

Facelift surgery is conventionally performed for the purpose of removing wrinkles from skin in cosmetic surgery and the like. The facelift surgery is surgery in which stretched and loose muscles and skin are lifted by removing parts thereof. The muscles lifted by the facelift surgery loosen again with a lapse of time. Performing the facelift surgery a plurality of times may have the risk of tightened skin in a face owing to reduced play of the facial muscles. Furthermore, there is a problem in a recovery period of the surgery in which, since operation portions bleed internally, women, in particular, hesitate to go out.

Patent Literature 1 proposes a facial wrinkle reduction device that includes a shaft having a proximal end portion and a distal end portion, means for dissolving tissue, and means that is connected to the shaft and provides energy to the target tissue.

### CITATION LIST

### Patent Literature

Patent Literature 1: Japanese Translation of PCT Application Publication No. 2003-523799

### SUMMARY OF INVENTION

### Technical Problem

However, since the facial wrinkle reduction device described in Patent Literature 1 removes tissue, there is also a concern for adverse effects on living bodies.

The present invention provides a biological stimulation device that can stimulate muscles to recover tension with no adverse effects on living bodies.

### Means for Solving Problem

A biological stimulation device of the present invention includes:

a stimulation device main body;

a heating part integrally provided at a distal end portion of the stimulation device main body; and

a first electrode part integrally provided at the distal end portion of the stimulation device main body.

### Advantageous Effects of the Invention

In the biological stimulation device of the present invention, the heating part and the first electrode part are integrally provided at the distal end portion of the stimulation device main body. Passing a current from the first electrode part via the skin through the living body can activate electron transport systems of mitochondria present in nerve cells to thereby promote the synthesis of ATP (adenosine triphosphate). The transportation of ions to the inside and outside of the cells can be smoothly achieved, and the excretion of waste products and carbon dioxide and the absorption of nutrition and oxygen can be intensified. This allows the promotion of metabolism of the nerve cells and the smoothness of repair, division, and reproduction.

Furthermore, passing a current from the first electrode part via a limited portion of the skin through the living body can activate the thermoreceptors.

In a state that the nerve cells and the thermoreceptors are activated by passing a current from the first electrode part of the biological stimulation device, as described above, a thermal stimulus is applied to the skin from the heating part, thereby stimulating the plurality of thermoreceptors. As a result, a stimulus to the nerve cells connected to the thermoreceptors is sufficiently increased, and a stimulus to the muscles transmitted from the nerve cells is increased. Therefore, the tension of the muscles is effectively recovered, thus allowing a reduction or removal of looseness, owing to, for example, a sag of the muscles for facial expression appearing on the face, and the like.

To be more specific, in a state of activating the nerve cells and the thermoreceptors by passing the current from the first electrode part of the biological stimulation device, as described above, when the thermal stimulus is applied from the heating part to the limited portion of the skin, neural transmission is smoothly performed and reliably transmitted to the muscles through the nerve cells of a user who requires an anti-aging treatment. In the muscles that receive the neural transmission, the tension of the muscle fibers constituting the muscles is improved, and metabolism of skin tissue is improved. As a result of improvement in blood circulation and metabolism of the skin, the tension (elasticity) of the loose muscles (e.g., the muscles for facial expression in the case of a face) is improved, and beautiful and tight skin can be obtained. For example, in the case of a face, the tight muscles for facial expression without looseness can be obtained.

In a state that the nerve cells and the thermoreceptors are activated by passing a current from the first electrode part of the biological stimulation device, as described above, when a thermal stimulus is applied from the heating part to a limited portion of the skin, the thermal stimulus from the skin to the peripheral nerve is smoothly transmitted to the nerve center, and serves to smoothly secrete brain transmitters having analgesic properties, inhibitory neurotransmitters, and the like, thus exerting a good pain relieving action. The hypertonicity of the sympathetic nerve is relieved, and vasoconstriction and muscle tonus are prevented, so that a good pain-relieving action is exerted.

Relaxing the muscle tonus allows the bones that have been pulled by the strained muscle and out of balance of positions (skeletal frame) to move back to their correct positions, and therefore exerts a good pain-relieving action for cervical spondylosis or pain caused by an imbalance of a skeletal frame.

Furthermore, when the heating part of the biological stimulation device is configured to repeatedly perform heating a plurality of times at predetermined time intervals, firing is effectively generated in the nerve cells by temporal summation, which is a feature of neural transmission. Therefore, an action potential can be effectively transmitted between the nerve cells. Accordingly, a stimulus transmitted from the nerve fibers to the muscles is further increased, thus allowing a more effective recovery of the tension of the muscles.

### BRIEF DESCRIPTION OF THE DRAWINGS

FIG. 1 is a perspective view illustrating a biological stimulation device.

FIG. 2 is a cross-sectional view of the biological stimulation device of FIG. 1.

FIG. 3 is a perspective view illustrating a heating part and a first electrode part.

FIG. 4 is a cross-sectional view illustrating a distal end portion of a stimulation device main body.

FIG. 5 is a perspective view illustrating usage of the biological stimulation device.

FIG. 6 is a perspective view illustrating another example of the biological stimulation device.

### DESCRIPTION OF EMBODIMENTS

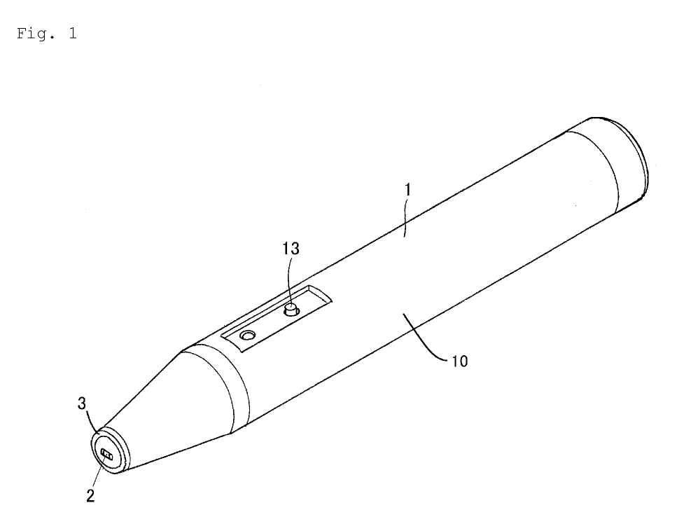

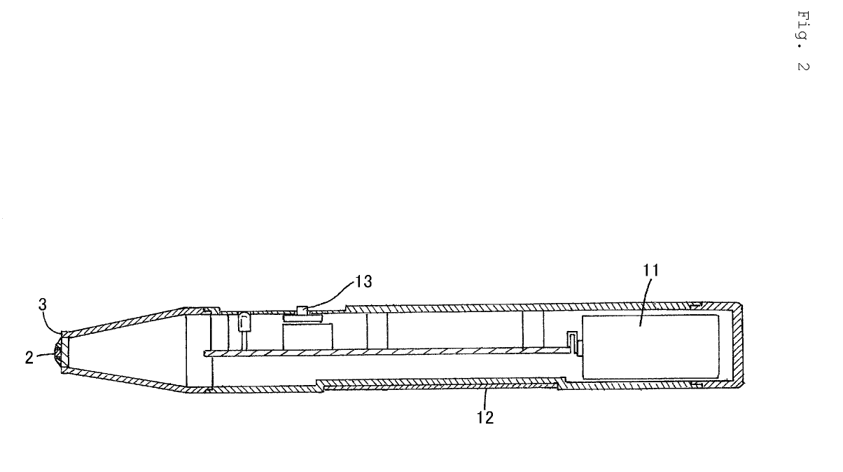

An example of a biological stimulation device of the present invention will be described with reference to the drawings. As illustrated in FIGS. 1 to 3, a biological stimulation device A includes a stimulation device main body 1, a heating part 2 integrally provided at a distal end surface of the stimulation device main body 1, and a first electrode part 3 integrally provided at the distal end surface of the stimulation device main body 1.

The stimulation device main body 1 is formed in such a size that a user can grip the stimulation device main body 1 with his or her one hand. In FIG. 1, the stimulation device main body 1 is in the shape of a cylinder having a certain length. The stimulation device main body 1 contains a direct current power supply 11 such as a dry battery or a secondary battery. Furthermore, a second electrode part 12 formed of an electroconductive material such as metal is integrally provided in a part of an outer peripheral surface of a grip portion 10 at which the user grips the stimulation device main body 1 with his or her hand. The second electrode part 12 is paired with the first electrode part 3 as an electrode unit. A negative electrode (positive electrode) of the direct current power supply 11 is electrically connected to the second electrode part 12, while a positive electrode (negative electrode) of the direct current power supply 11 is electrically connected to the first electrode part 3, which will be described later. A switch 13 to turn on or off the biological stimulation device A is provided in the outer peripheral surface of the stimulation device main body 1.

Instead of the direct current power supply 11 contained in the stimulation device main body 1, an outside direct current power supply may be used without the provision of the direct current power supply 11 therein.

The distal end surface of the stimulation device main body 1 protrudes from an outer peripheral portion toward a central portion (convex arc shape in section), and the single heating part 2 is integrally provided at the central portion of the distal end surface. Note that a plurality of heating parts 2 may be integrally provided in the distal end surface of the stimulation device main body 1. The distal end surface of the stimulation device main body 1 is not necessarily formed to have the convex arc shape in section, but may be formed into a flat surface and have one or a plurality of heating parts at a central portion of the flat distal end surface.

The heating part may protrude from the distal end surface of the stimulation device main body 1, or a distal end surface of the heating part may be flush with the distal end surface of the stimulation device main body 1.

By heating the heating part 2 and bringing the heated heating part 2 into contact with a skin surface of a living body, such as a human body, a thermal stimulus is applied to the skin of the living body. Note that, although the heating part 2 may be covered with a covering member of such as a synthetic resin as long as the heating part 2 can apply a thermal stimulus to the living body, the heating part 2 is preferably exposed.

The size of the distal end surface of the heating part 2 is preferably 0.5 to 3 mm, and more preferably 1 to 2 mm, because, while the skin of the living body is pressed to thin the skin and subcutaneous tissue, effectively applying a thermal stimulus to the nerve cells effectively generates firing in the nerve cells by temporal summation. Thus, an action potential is effectively transmitted between the nerve cells, and firing is effectively generated in the nerve cells, so that it is possible to increase a stimulus transmitted from the nerve fibers to the muscles and therefore effectively recover the tension of the muscles. Note that the size of the heating part 2 is a minimum diameter of a perfect circle that can enclose the heating part 2.

The heated temperature of the heating part 2 is preferably 43 to 65° C., and more preferably 48 to 60° C., though it depends on each living body. This temperature range makes it possible to effectively generate firing by temporal summation in the nerve cells, and effectively transmit an action potential between the nerve cells and generate firing in the nerve cells, thus allowing an increase in a stimulus transmitted from the nerve fibers to the muscles and an effective recovery of the tension of the muscles.

The heating part 2 is not specifically limited as long as it can be heated to a predetermined temperature by energization. A heating time of the heating part 2 is preferably controlled by control of energization. Examples of the heating part 2 include a nichrome wire, and a ceramic chip. A nichrome wire or ceramic chip is electrically connected to the direct current power supply 11, and can be heated to a predetermined temperature by feeding a direct current to rise its temperature. The direct current fed through the nichrome wire or the ceramic chip can be controlled to control the heated temperature and the heated time of the nichrome wire or the ceramic chip.

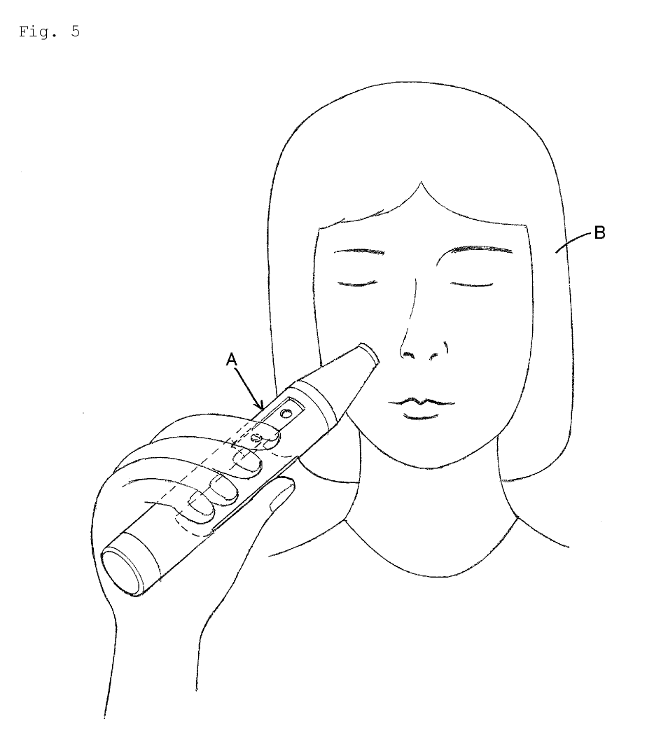

To be more specific, as illustrated in FIG. 4, a pair of electrical connection portions 2A and 2B are disposed at the central portion of the distal end surface of the stimulation device main body 1 in such a state as to expose distal end surfaces of the electrical connection portions 2A and 2B. Between the electrical connection portions 2A and 2B, the heating part 2 such as a nichrome wire or a ceramic chip is disposed in an electrically connected manner. Note that the electrical connection portion 2A is electrically connected to the positive electrode or the negative electrode of the direct current power supply contained in the stimulation device main body 1, while the electrical connection portion 2B is electrically connected to the negative electrode or the positive electrode of the direct current power supply.

When the distal end surface of the stimulation device main body 1 is pressed against a user's skin, the electrical connection portions 2A and 2B are in contact with the skin, and a weak current of the order of 30 to 150 μA passes through the living body of the user that is present between the electrical connection portions 2A and 2B. Accordingly, as will be described later, while the heating part 2 is heated to a predetermined temperature by feeding a direct current into the heating part 2, the weak current passes through the living body of the user. The passing of the weak current through the living body from the electrical connection portions 2A and 2B has the same effects as the passing of a weak current through the living body from the electrode part 3, which will be described later. A specific description of the effects is omitted here with the aid of a description about the case of passing a weak current through the living body from the electrode part 3.

The passing of a weak current from the electrical connection portions 2A and 2B through the living body is constantly performed concurrently with a thermal stimulus by the heating part 2. After the nerve cells and the thermoreceptors are activated by passing the weak current, the nerve cells are effectively electrically and thermally stimulated to effectively generate firing in the nerve cells. Then, the muscles connected to the nerve fibers are effectively stimulated, to cause the muscle fibers to recover flexibility and elasticity, and provide tension to the muscles. As a result, it is possible to effectively reduce or remove wrinkles, looseness of the muscles, and the like appearing on the skin.

In a state that the nerve cells and the thermoreceptors are activated by passing a weak current from the electrical connection portions 2A and 2B through the living body, if a thermal stimulus is applied from the heating part to the skin, the thermal stimulus from the skin to the peripheral nerves is smoothly transmitted to the nerve center, and serves to smoothly secrete brain transmitters (β-endorphin and enkephalin) having analgesic properties, inhibitory neurotransmitters (γ-aminobutyric acid), and the like, thus exerting a good pain relieving action. Furthermore, the hypertonicity of the sympathetic nerve is relieved, and vasoconstriction and muscle tonus are prevented, so that a good pain-relieving action is exerted.

Furthermore, preventing the vasoconstriction and the muscle tonus promotes looseness of the muscles. Relaxing the muscles allows the bones that have been pulled by the strained muscles and have deviated from the correct positions to move back to their correct positions. Moving back the bones to the correct positions exerts a good pain-relieving action.

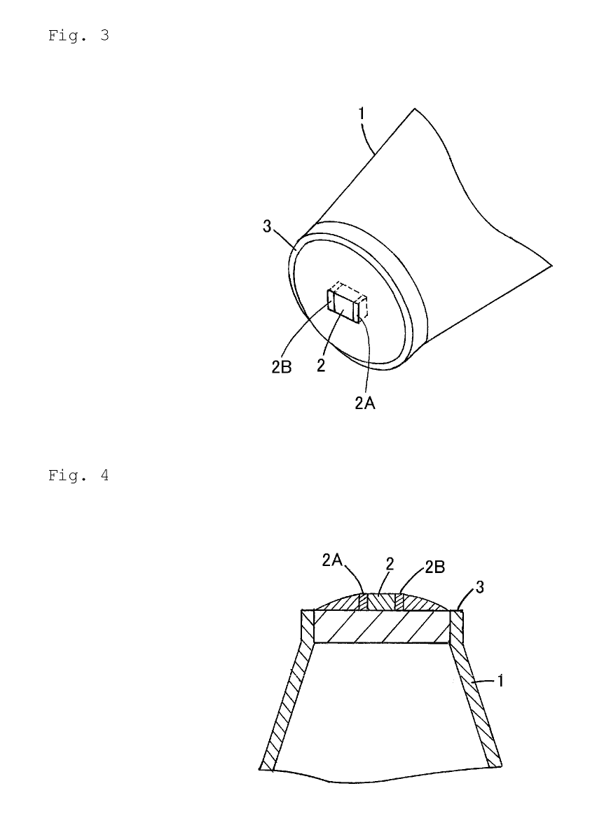

The ring-shaped first electrode part 3 is integrally provided in an outer peripheral portion of the distal end surface of the stimulation device main body 1, so as to enclose the heating part 2. The first electrode part 3 is formed of an electroconductive material. In FIGS. 3 and 4, the first electrode part 3 is formed in a ring shape, but the ring-shaped first electrode part 3 may be divided into a plurality of portions. Each divided portion of the first electrode part is electrically connected to the direct current power supply 11. The biological stimulation device is configured such that, when the distal end surface of the stimulation device main body 1 is pressed against a user's skin, the distal end surface of the stimulation device main body 1 pushes the skin at its protruding portion, so that both the heating part 2 and the first electrode part 3 are brought into contact with the skin.

As described above, the first electrode part 3 is electrically connected to the positive electrode or the negative electrode of the direct current power supply 11. Bringing the first electrode part 3 into contact with the skin surface of the living body such as a human body, can form an electrical circuit that connects the positive electrode (negative electrode) of the direct current power supply 11, the first electrode part 3, a human body portion (for example, face) with which the first electrode part 3 is in contact, the abdomen, the arm and the palm griping the stimulation device main body, the second electrode part 12 of the stimulation device main body 1, and the negative electrode (positive electrode) of the direct current power supply 11. A weak current supplied from the direct current power supply 11 is applied to the nerve cells in the living body. The weak current is preferably 1 to 300 μA, and more preferably 1 to 150 μA.

Turning on (powering on) of the power supply by a press of the switch 13 of the biological stimulation device A can bring a state in which a direct current from the direct current power supply 11 is constantly fed through the first electrode part 3, while a direct current from the direct current power supply 11 is fed to the heating part 2 at predetermined time intervals so as to heat the heating part 2 to a predetermined temperature at predetermined time intervals.

Energization control means, which are contained in the biological stimulation device A, controls the feeding of the direct currents into the first electrode part 3 and the heating part 2. More specifically, the stimulation device main body 1 contains a CPU (central processing unit). To the CPU, a ROM (read only memory) and a RAM (random access memory) are electrically connected so as to be able to transmit and receive electrical signals. The ROM stores an energization control program executed by the CPU and various types of data. The RAM stores a memory for temporarily storing setting values set on the basis of the energization control program, and the like.

Note that the energization control means is implemented by causing the CPU and the RAM to read and store the energization control program, and data to be read from and written in the RAM under control of the CPU.

The stimulation device main body 1 may be provided with a heating switch (not illustrated), and the heating part 2 may be heated to a predetermined temperature at predetermined time intervals only while the heating switch is pressed.

A direct current feed (supply) pattern by the CPU into the heating part 2 is not specifically limited. However, the supply pattern is preferably constituted of repetitions of a cycle provided that one cycle includes the step of supplying a direct current to the heating part 2 of the biological stimulation device A for a predetermined time period (for example, 0.2 to 1.2 seconds) and thereafter the step of stopping the supply of the direct current for a predetermined time period (for example, 0.1 to 1.2 seconds).

Upon feeding the direct current into the heating part 2, the heating part 2 is instantaneously heated to a predetermined temperature. On the other hand, upon stopping the feeding of the direct current into the heating part 2, the heating of the heating part 2 is stopped, and the temperature of the heating part 2 drops.

Accordingly, the above-described direct current supply pattern repeatedly performs the cycle in which the heating part 2 is heated to the predetermined temperature for the predetermined time period by the supply of the direct current, and thereafter the heating is stopped for the predetermined time period in accordance with the stop of the direct current to drop the temperature.

The heating time of the heating part 2 is preferably 0.3 to 1 second, and more preferably 0.4 to 0.7 seconds, because firing can be effectively generated in the nerve cell by temporal summation.

The time for stopping supplying the direct current to the heating part 2 is preferably 0.1 to 0.8 seconds, and more preferably 0.2 to 0.6 seconds, because intermittently and repeatedly applying a thermal stimulus to the nerve cells allows the effective generation of firing in the nerve cells by temporal summation.

Next, usage of the biological stimulation device A will be described. Upon turning on the biological stimulation device A by a push of the switch 13 of the biological stimulation device A, a direct current is constantly fed into the first electrode part 3 of the biological stimulation device A, while a direct current is fed into the heating part 2 at predetermined time intervals, in order to heat the heating part 2 to a predetermined temperature at predetermined time intervals, under control of the CPU in accordance with the energization control program.

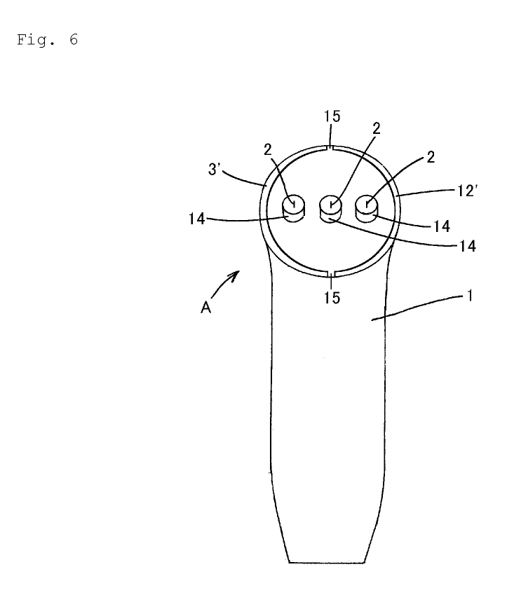

As illustrated in FIG. 5, a person (hereinafter referred to as “user”) B, who wants to recover tension by providing stimuluses to the muscles or who wants to reduce pain, grips the grip portion 10 of the stimulation device main body 1 with his or her hand, and presses the distal end surface of the biological stimulation device A against the skin of the user himself or herself. Note that the user grips the biological stimulation device A in such a manner that part of his or her palm or finger is in contact with the second electrode part 12 of the stimulation device main body 1, so that the palm or finger is electrically connected to the second electrode part 12.

When the first electrode part 3 that is integrally provided in the distal end surface of the biological stimulation device A is brought into contact with the skin and a weak current is allowed to pass through the living body via the first electrode part 3, electron transport systems of the mitochondria present in the nerve cells are activated, and the generation of the mitochondria themselves is increased, so that the synthesis of ATP (adenosine triphosphate) is promoted. Furthermore, since the generation of ribosome RNA is increased, the repair of tissue owing to the activation of synthesis of protein is promoted. As a result, the transportation of ions to the inside and outside of the cells can be smoothly performed, and the excretion of waste products and carbon dioxide and the absorption of nutrition and oxygen can be intensified. This allows the promotion of metabolism of the nerve cells and the smoothness of repair, division, and reproduction, thereby activating the nerve cells.

In addition, passing the weak current through the living body via the first electrode part 3 can smoothly generate firing in transmitting neurotransmitters from one nerve cell to the next nerve cell, and firing in the nerve cells.

Passing the weak current through the living body via the first electrode part 3 can activate the thermoreceptors, and increase the reaction of the thermoreceptors to the thermal stimulus from the heating part. A stimulus to the nerve cells connected to the thermoreceptors is thereby sufficiently increased, and a stimulus transmitted from the nerve cells to the muscles is increased. Therefore, it is possible to effectively recover the tension of the muscles, and reduce or remove looseness owing to, for example, a sag of the muscles for facial expression appearing on the face, and the like.

In a state that the nerve cells and the thermoreceptors are activated by passing the weak current via the first electrode part 3 through the living body as described above, when the thermal stimulus is applied from the heating part 2 to the skin, the thermal stimulus from the skin to the peripheral nerve is smoothly transmitted to the nerve center, and serves to smoothly secrete brain transmitters (β-endorphin and enkephalin) having analgesic properties, inhibitory neurotransmitters (γ-aminobutyric acid), and the like, thus exerting a good pain relieving action. Furthermore, the hypertonicity of the sympathetic nerve is effectively relieved, and the vasoconstriction and the muscle tonus are prevented, so that a good pain-relieving action is exerted.

Furthermore, preventing the vasoconstriction and the muscle tonus promotes looseness of the muscles. The looseness of the muscles allows the bones, that have been pulled by the strained muscles and have deviated from their correct positions, to move back to the correct positions. Moving back the bones to the correct positions exerts a good pain-relieving action.

As described above, in a state that the weak current is applied to the living body via the first electrode part 3 and the nerve cells and the thermoreceptors are activated, the thermal stimulus is applied to the skin of the user (living body) at predetermined time intervals from the heating part 2 of the biological stimulation device A that is pressed on the skin.

In other words, one cycle is defined to include a heating pattern in which after the heating part 2 is heated to a predetermined temperature for a predetermined time period by the supply of the direct current, the heating is stopped for a predetermined time period in accordance with stop of the direct current to drop the temperature, and the cycle is repeatedly performed.

The first electrode part 3 is disposed so as to enclose the heating part 2. Since the nerve cells and the thermoreceptors to which the heating part 2 provides the thermal stimulus are in an activated state owing to the weak current supplied through the first electrode part 3, the thermal stimulus by the heating part 2 is reliably applied to the nerve cells and the thermoreceptors in the activated state.

An information transmission mechanism in the nerve fibers will be described. The nerve fiber is constituted of an infinite number of nerve cells. The nerve cell has a cell body, a plurality of dendrites that are split from the cell body like branches, and an axon protruding from the cell body.

The nerve cell transmits information using signals. When the cell body is in a resting state, while many potassium ions are present in the nerve cell, sodium ions and chlorine ions are widely distributed outside the nerve cell. The inside of the nerve cell thereby maintains a minus potential (resting membrane potential) with respect to the outside of the nerve cell. The difference in concentration of the potassium ions, the sodium ions, and the chlorine ions is maintained by sodium pumps of transmembrane protein of the nerve cell, which sodium pumps use ATP as energy. The passing of a weak current through a human body promotes the synthesis of ATP, which is used as energy.

On the other hand, when a signal is transmitted in the nerve cell, pores called sodium channels present in a cell membrane are opened instantaneously for about one thousandth of a second, so that sodium ions flow into the nerve cell therethrough. As a result, the potential of the inside of the nerve cell slightly becomes positive (action potential) with respect to the outside of the cell body. This phenomenon is called “firing”. The potential of the inside of the nerve cell gradually increases, and upon the potential exceeds a certain threshold value, the action potential is generated in a stroke. As will be described later, the “firing” is generated when a physical or chemical stimulus is received from the outside, as well as when neurotransmitters are received from another nerve cell.

Usually, the “firing” is first generated at a base of the axon extending from the cell body, in other words, an axon hillock. The generated action potential brings about an electrical change in the potential of the cell membrane of the nearby axon. When the potential change of the membrane exceeds the predetermined threshold value, the action potential is sequentially transmitted through the axon to the axonal terminal.

Next, the action potential transmitted to the axonal terminal is transmitted to the next nerve cell. Since a slight gap (synaptic gap) is present between the axonal terminal of the nerve cell and the dendrite of the next nerve cell, the action potential cannot be transmitted to the next nerve cell in the above-described way.

The action potential transmitted to the axonal terminal opens calcium channels of the synaptic terminal, so that calcium ions flow into the synaptic terminal therethrough. When neurotransmitters are released from the synaptic vesicles present inside the synaptic terminal and connected to the dendrite of the next nerve cell, as described above, the sodium channels are opened, and an action potential is generated in the next nerve cell and transmitted through the nerve cell. Although it is required to transmit neurotransmitters from the one nerve cell to the next nerve cell and generate “firing” in the next nerve cell, the generation of firing requires the action potential to be a predetermined threshold value or more.

For example, in the youth of twenty-to-thirtysomethings, firing is easily generated in the nerve cells, when neurotransmitters are transmitted from one nerve cell to the next nerve cell, or when a physical or chemical stimulus is received from the outside. However, as age increases, firing is hard to generate, and it is often the case that transmission from one nerve cell to the next nerve cell is not smoothly performed, or the firing itself is not smoothly generated in the nerve cell.

The biological stimulation device A can promote the generation of ATP by supplying a weak current from the first electrode part 3, and serve to smoothly perform transmission from one nerve cell to the next nerve cell and the generation of firing itself in the nerve cell.

In a nerve cell, a phenomenon (temporal summation) in which repeatedly applying repetitive stimuluses, such as heat, at sufficiently short time intervals adds and increases an action potential generated in the nerve cell occurs.

In the biological stimulation device A, in order to effectively generate firing in the case of transmitting neurotransmitters from one nerve cell to the next nerve cell, or firing in the case of applying a physical or chemical stimulus to the nerve cells from the outside, a weak current is applied to the nerve cells through the first electrode part 3 to activate the nerve cells and the thermoreceptors and effectively generate firing in the nerve cells, while a thermal stimulus is applied from the heating part 2 to the activated nerve cells and thermoreceptors to preferably generate temporal summation. As a result, the effective generation of the firing in the nerve cells and the effective provision of the stimulus through the nerve fibers to the muscles make the muscle fibers have flexibility and recovered elasticity.

In other words, the biological stimulation device A repeatedly applies the thermal stimulus to the same nerve cells under skin at the predetermined time intervals, while activating the nerve cells by the weak current, to concurrently generate temporal summation owing to the thermal stimulus. As a result, the firing is effectively generated in the nerve cells, and the action potential is effectively transmitted through the nerve cells. As a result, it is possible to effectively stimulate the muscles, and therefore make the muscle fibers have flexibility and recovered elasticity.

As described above, in the biological stimulation device A, the first electrode part 3 and the heating part 2 are disposed in the distal end surface of the stimulation device main body 1. Accordingly, even if a user is not skilled, the user can press the first electrode part 3 and the heating part 2 against a plurality of thermoreceptors converging at the same nerve cell, with a broad understanding of a path of the nerve fibers, only by pressing the distal end surface of the stimulation device main body 1 on his or her skin. In a state that the nerve cells and the thermoreceptors are activated by a weak current supplied through the first electrode part 3, the first electrode part 3 and the heating part 2 effectively apply electrical and thermal stimuluses to the nerve cells, in order to effectively generate firing in the nerve cells and effectively stimulate the muscles connected to the nerve fibers. Therefore, since the muscle fibers have flexibility and recovered elasticity, the muscles have tense, so that wrinkles, looseness of the muscles, and the like appearing on the skin can be effectively reduced or removed.

In the biological stimulation device A, the first electrode part 3 is constantly energized, but the first electrode part 3 is not necessarily constantly energized as long as the first electrode part 3 is energized while a thermal stimulus is applied to the nerve cells by energization of the heating part 2. For example, the first electrode part may be energized in the same timing as the energization to the heating part 2, to apply a weak current to the nerve cells.

In the biological stimulation device A, the first electrode part 3 is provided in the distal end surface of the stimulation device main body 1, and the second electrode part 12 is provided in the grip portion 10 of the stimulation device main body 1, but both a first electrode part 3′ and a second electrode part 12′ may be provided in the distal end surface of the stimulation device main body 1. It is not required to provide the second electrode part 12 in the grip portion 10 of the stimulation device main body 1. The same components as those of the above-described biological stimulation device A are indicated with the same reference numerals and a description thereof is omitted. A stimulation device main body 1 is provided with a switch for powering on and off a power supply, though the switch is not illustrated in FIG. 6. A direct current power supply 11 may be contained in the stimulation device main body 1, but an outside direct current power supply may be used instead without containing the direct current power supply.

To be more specific, a half ring-shaped first electrode part 3′ is integrally provided in an outer peripheral portion of a first half (left half in FIG. 6) of a distal end surface of the stimulation device main body 1. A half ring-shaped second electrode part 12′ is integrally provided in an outer peripheral portion of a second half (right half in FIG. 6) of the distal end surface of the stimulation device main body 1. In the distal end surface of the stimulation device main body 1, gap portions 15 are present between opposite surfaces of the first electrode part 3′ and the second electrode part 12′, so that the first electrode part 3′ and the second electrode part 12′ are electrically insulated. The second electrode part 12′ is paired with the first electrode part 3′ as an electrode unit. The positive electrode (negative electrode) of the direct current power supply 11 is electrically connected to the first electrode part 3′, and the negative electrode (positive electrode) of the direct current power supply 11 is electrically connected to the second electrode part 12′. The first electrode part 3′ and the second electrode part 12′ are formed of an electroconductive material. Each of the first electrode part 3′ and the second electrode part 12′ may be divided into a plurality of portions. Each divided portion of the first electrode part 3′ and the second electrode part 12′ is electrically connected to the direct current power supply 11.

The first electrode part 3′ and the second electrode part 12′ are disposed opposite to each other with respect to heating parts 2 as a center. The first electrode part 3′ and the second electrode part 12′ enclose the heating parts 2.

The heating parts 2 are integrally provided at a central portion of the distal end portion of the stimulation device main body 1. As in the case of the biological stimulation device A of FIG. 1, the heating parts 2 may be integrally provided in the distal end surface of the stimulation device main body 1. However, as illustrated in FIG. 6, one or a plurality of columnar heating part bases 14 may be integrally provided in the distal end surface of the stimulation device main body 1, and the heating parts 2 may be integrally provided on distal end surfaces of the heating part bases 14. In this case, the height of the heating part bases 14 is required to be adjusted such that, when a distal end portion of the stimulation device main body 1 is pressed against the skin, the first electrode part 3′ and the second electrode part 12′, as well as the heating parts 2, can be brought into contact with the skin.

The area of the distal end surface of the heating part base 14 is preferably 2 to 40 mm², more preferably 2 to 30 mm², more preferably 2 to 20 mm², more preferably 2 to 10 mm², more preferably 3 to 7 mm², and particularly preferably 3 to 5 mm². This is because effectively applying a thermal stimulus to the nerve cells, while the skin and subcutaneous tissue are thinned by pressing the skin of the living body, effectively generates firing in the nerve cells by temporal summation, and effectively transmits an action potential between the nerve cells and generates firing inside the nerve cells, so that an increase in a stimulus transmitted from the nerve fibers to the muscles serves to effectively recover the tension of the muscles.

The distal end surface of the heating part base 14 protrudes from an outer peripheral portion toward a central portion (convex arc shape in section), and the single heating part 2 is integrally provided at the central portion of the distal end surface of the heating part base 14. Note that a plurality of heating parts 2 may be integrally provided in the distal end surface of the heating part base 14. The distal end surface of the heating part base 14 is not necessarily formed to have the convex arc shape in section, but may be formed into a flat surface and have one or a plurality of heating parts 2 formed at a central portion of the flat distal end surface.

The heating part 2 may protrude from the distal end surface of the heating part base 14, or a distal end surface of the heating part 2 may be flush with the distal end surface of the heating part base 14. The heating part 2 may be covered with a covering member of such as a synthetic resin as long as the heating part 2 can apply a thermal stimulus to the living body, but the heating part 2 is preferably exposed. The structure and disposition of the heating part 2 and electrical connection portions 2A and 2B in the distal end surface of the heating part base 14 are the same as those of the heating part 2 and the electrical connection portions 2A and 2B in the distal end surface of the stimulation device main body 1 described above, and so a description thereof is omitted.

Usage of the biological stimulation device A configured as described above will be described. A user B grips the stimulation device main body 1, and presses the distal end portion of the biological stimulation device A against the skin of the user himself or herself. In this state, the heating part bases 14 of the biological stimulation device A push the skin, and the heating parts 2 provided at the distal end surfaces of the heating part bases 14, the first electrode part 3′, and the second electrode part 12′ are in contact with the skin.

A weak current supplied from the direct current power supply 11 passes through the user's living body present between the first electrode part 3′ and the second electrode part 12′, so that the weak current is applied to the nerve cells in the living body. The weak current is preferably 1 to 300 μA, and more preferably 1 to 150 μA.

The biological stimulation device A passes the weak current through the user's living body present between the first electrode part 3′ and the second electrode part 12′. However, also in this case, by the same actions as the biological stimulation device A of FIGS. 1 to 4, it is possible to activate the nerve cells and the thermoreceptors, and effectively generate firing in the nerve cells. By applying thermal stimuluses from the heating parts 2 to the user's (living body) skin, as in the case of the biological stimulation device A of FIGS. 1 to 4, the nerve cells are effectively electrically and thermally stimulated, and firing is effectively generated in the nerve cells. Therefore, the muscles connected to the nerve cells are effectively stimulated, so that it is possible to effectively reduce or remove wrinkles, looseness of the muscles, and the like appearing on the skin. Furthermore, passing a weak current through the user's living body present between the first electrode part 3′ and the second electrode part 12′ exerts a good pain-relieving action, as in the case of the biological stimulation device A of FIG. 1.

In the above-described biological stimulation device A, the first electrode part 3′ and the second electrode part 12′ are disposed opposite to each other with respect to the heating parts 2 as the center, and the heating parts 2 are enclosed by the first electrode part 3′ and the second electrode part 12′. Since the nerve cells and the thermoreceptors to which the heating parts 2 provide the thermal stimuluses are put in an activated state owing to the weak current supplied from the first electrode part 3′ and the second electrode part 12′, the thermal stimuluses are reliably applied from the heating parts 2 to the nerve cells and the thermoreceptors in the activated state.

A way to pass the weak current through the living body of the user present between the first electrode part 3′ and the second electrode part 12′ (a way to pass the weak current between the first electrode part 3′ and the second electrode part 12′) is the same as that of the biological stimulation device A illustrated in FIGS. 1 to 4, and so a description thereof is omitted. A way to heat the heating parts 2 and a direct current feed (supply) pattern by a CPU into the heating parts 2 are the same as those of the biological stimulation device A illustrated in FIGS. 1 to 4, and so descriptions thereof are omitted.

### INDUSTRIAL APPLICABILITY

In the biological stimulation device of the present invention, in a state that a weak current is applied from the electrode unit to the nerve cells to activate the nerve cells and the thermoreceptors, the thermoreceptors are stimulated by a thermal stimulus from the heating part. Since a stimulus to the nerve cells connected to the thermoreceptors is sufficiently increased and a stimulus transmitted from the nerve cells to the muscles is increased, it is possible to effectively recover the tension of the muscles. The biological stimulation device of the present invention smoothly promotes the secretion of brain transmitters having analgesic properties, inhibitory neurotransmitters, and the like. The biological stimulation device of the present invention effectively relieves the hypertonicity of the sympathetic nerve, and prevents the vasoconstriction and muscle tonus, thus exerting a good pain-relieving action.

Furthermore, the suppression of the vasoconstriction and the muscle tonus can promote looseness of the muscles. The looseness of the muscles can move back the bones to their correct positions, and therefore alleviate pain owing to displacement of the bones.

The biological stimulation device of the present invention can be favorably used for a pain alleviation treatment for a human body, as well as used for a cosmetic application to reduce or remove looseness owing to, for example, a sag of the muscles of facial expression appearing on a face, and the like.

### CROSS-REFERENCE TO RELATED APPLICATION

The present application claims the priorities under Japanese Patent Application No. 2016-132836 filed on Jul. 4, 2016 and Japanese Patent Application No. 2016-152895 filed on Aug. 3, 2016, the disclosures of which are hereby incorporated in their entirety by reference.

### REFERENCE SIGNS LIST

1 stimulation device main body
2 heating part
21 covering member
3, 3′ first electrode part
11 direct current power supply
12, 12′ second electrode part
13 switch
A biological stimulation device

1. A biological stimulation device comprising:
a stimulation device main body;
a heating part integrally provided at a distal end portion of the stimulation device main body; and
a first electrode part integrally provided at the distal end portion of the stimulation device main body.

2. The biological stimulation device according to claim 1, wherein the first electrode part is provided to enclose the heating part.

3. The biological stimulation device according to claim 1, further comprising a second electrode part that is integrally provided in a grip portion of the stimulation device main body and paired with the first electrode part.

4. The biological stimulation device according to claim 1, further comprising a second electrode part that is integrally provided at the distal end portion of the stimulation device main body and paired with the first electrode part.

5. The biological stimulation device according to claim 4, wherein the heating part is enclosed by the first electrode part and the second electrode part.

6. The biological stimulation device according to claim 4, wherein the first electrode part and the second electrode part are disposed opposite to each other with respect to the heating part as a center.

7. The biological stimulation device according to claim 1, wherein the biological stimulation device is configured such that the heating part is heated when the electrode part is supplied with a current.

8. The biological stimulation device according to claim 1, wherein the biological stimulation device is configured such that the heating part is repeatedly heated a plurality of times at predetermined time intervals.
